(19) United States
(12) Patent Application Publication
(10) Pub. No.: US 2020/0188156 A1
(43) Pub. Date: Jun. 18, 2020

(54) Knee Brace

(71) Applicant: Eric Michael Rippetoe, Shreveport, LA (US)

(72) Inventor: Eric Michael Rippetoe, Shreveport, LA (US)

(21) Appl. No.: 16/712,860

(22) Filed: Dec. 12, 2019

Related U.S. Application Data
(60) Provisional application No. 62/778,592, filed on Dec. 12, 2018.

(51) Int. Cl. A61F 5/01

(52) U.S. Cl. CPC A61F 5/0123; A61F 2005/0137

(57) ABSTRACT

The present invention is a knee brace includes a thigh sleeve that completely wrap around part of the thigh, a calf sleeve that completely wrap around part of the calf, and a pair of double hinges that positioned on both lateral and medial side of the knee. The thigh sleeve and the calf sleeve are removably mounted to each other through the pair of double hinges. The double hinges positioned on both side of the knee enables the user to move their leg without hinderance, but also prevent the user from hyperextending the knee in unwanted or damaging direction.

The current application claims a priority to the U.S. Provisional Patent application Ser. No. 62/778,592 filed on Dec. 3, 2018.

### FIELD OF THE INVENTION

The present invention generally relates to a knee brace. More specifically, the present invention is an injury prevention knee brace that wrap around the user's thigh and calf but leave the knee open for unhindered movement.

### BACKGROUND OF THE INVENTION

As many people in the present society do not exercise as much as before due to sedentary jobs, obesity, or other causes, knee injury became more prevalent and prevent people from exercising. While many knee braces are available to facilitate users to exercise, many issues arise from the structure of the knee braces. Those knee braces are either too heavy, too complex to assemble, hard to wear or move, or simply hinder the user's leg movement. Moreover, the majority of those knee braces are not intended to be worn during exercise, thus they can only hinder, not facilitate the user's movement.

The present invention solves issues mentioned above by providing a knee brace that covers both thigh and calf but leave both front and the back of the knee open. The present invention has hinges near the knee to facilitate and protect the user's leg from bending in unwanted angles or directions. The present invention is very easy to assemble, as the user may choose to wear the thigh component and the calf component separately, then join the rest of the present invention with hinges near the knee. Finally, the present invention is light-weighted but durable, which allows the user to wear during exercise without worrying about hindering the leg movement while capable of preventing injury.

### BRIEF DESCRIPTION OF THE DRAWINGS

FIG. 1 is a perspective view of the present invention.

FIG. 2 is a perspective view of the present invention, showing the fastening ends.

FIG. 3 is a perspective view of the present invention, showing the configuration of the thigh strap assembly and the calf strap assembly.

FIG. 4 is a top perspective view of the present invention showing the thigh sleeve and the calf sleeve.

FIG. 5 is a bottom perspective view of the present invention showing thigh sleeve and the calf sleeve.

FIG. 6 is an exploded view of the hinge section between the thigh strap assembly and the calf strap assembly of the present invention.

FIG. 7 is a front perspective view of the present invention showing the knee opening.

FIG. 8 is a rear perspective view of the present invention showing the joint opening.

FIG. 9 is a side view of the present invention showing the upper and lower lateral fastening ends.

FIG. 10 is a side view of the present invention showing the upper and lower medial fastening ends.

FIG. 11 is a front perspective view of the upper and lower lateral fastening ends and the upper and lower medial fastening ends of the present invention showing the locking position.

FIG. 12 is a front perspective view of the upper and lower lateral fastening ends and the upper and lower medial fastening ends of the present invention showing the unlocking position.

### DETAIL DESCRIPTION OF THE INVENTION

All illustrations of the drawings are for the purpose of describing selected versions of the present invention and are not intended to limit the scope of the present invention.

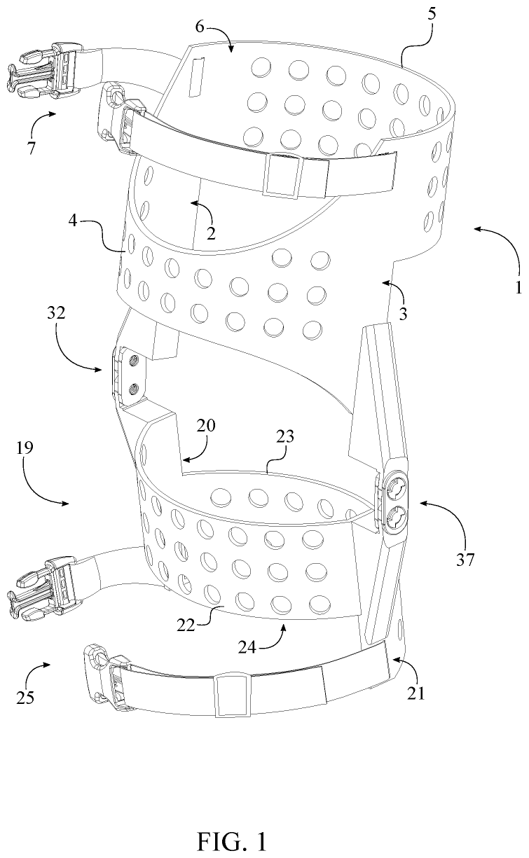

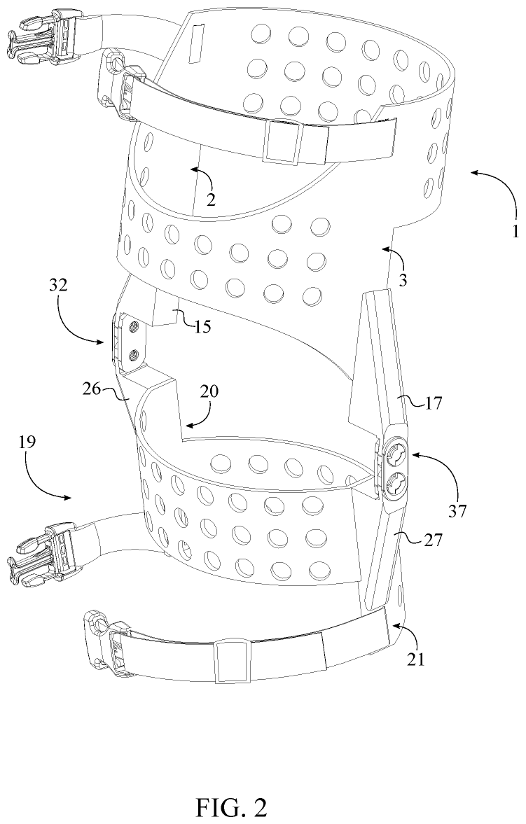

In reference to FIGS. 1 and 2, the present invention is a knee brace that wraps around the user's thigh and calf but leaves openings for the user's knee and joint (back of the knee) to ensure unhinged movement during exercise or other regular movements. The present invention comprises a thigh sleeve 1, a calf sleeve 19, a lateral fastening mechanism 32, and a medial fastening mechanism 37. The thigh sleeve 1 is configured to wrap around the part of user's thigh adjacent to the knee. The thigh sleeve 1 comprises an upper lateral side 2, an upper medial side 3, at least one thigh strap assembly 7, an upper lateral fastening end 15, and an upper medial fastening end 17. The upper lateral side 2 is positioned on the lateral side of the thigh sleeve 1 and is configured to protect the lateral side of the user's thigh. The upper medial side 3 is positioned on the medial side of the thigh sleeve 1 and is configured to protect the medial side of the user's thigh. The at least one thigh strap assembly 7 is adjustably integrated onto the upper lateral side 2 and the upper medial side 3. The user may tighten the thigh sleeve 1 with the at least one thigh strap assembly 7 by tightening the upper lateral side 2 and the upper medial side 3 to the user's thigh to prevent the thigh sleeve 1 from slipping off from the user's thigh.

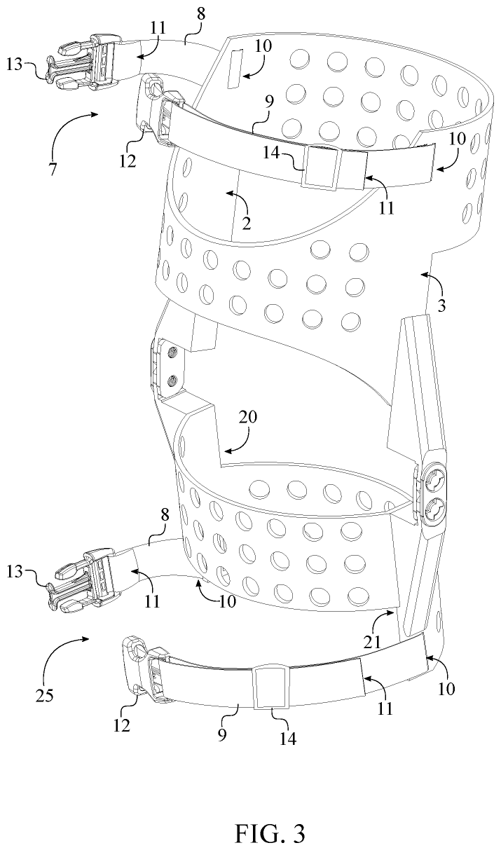

In reference to FIGS. 2 and 3, the calf sleeve 19 comprising a lower lateral side 20, a lower medial side 21, at least one calf strap assembly 25, a lower lateral fastening end 26, and a lower medial fastening end 27. The lower lateral side 20 is positioned on the lateral side of the calf sleeve 19 and is configured to protect the lateral side of the user's calf. The lower medial side 21 is positioned on the medial side of the calf sleeve 19 and is configured to protect the medial side of the user's call The at least one calf strap assembly 25 is adjustably integrated onto the lower lateral side 20 and the lower medial side 21. The user may tighten the calf sleeve 19 with the at least one calf strap assembly 25 by tightening the lower lateral side 20 and the lower medial side 21 to the user's calf to prevent the calf sleeve 19 from slipping off from the user's calf.

In reference to FIG. 2, the upper lateral fastening end 15 is terminally connected onto the upper lateral side 2. The upper lateral fastening end 15 is configured to connect between the upper lateral side 2 and the lateral fastening mechanism 32. The lower lateral fastening end 26 is terminally connected onto the lower lateral side 20. The lower lateral fastening end 26 configured to connect between the lower lateral side 20 and the lateral fastening mechanism 32. As a result, the upper lateral fastening end 15 and the lower lateral fastening end 26 are rotatably attached to each other by the lateral fastening mechanism 32. This configuration allows the user to change the lateral angle between the thigh sleeve 1 and the calf sleeve 19, as the user's knee changes the angle.

The upper medial fastening end 17 is terminally connected onto the upper medial side 3. The upper medial fastening end 17 is configured to connect between the upper medial side 3 and the medial fastening mechanism 37. The lower medial fastening end 27 is terminally connected onto the lower medial side 21. The lower medial fastening end 27 configured to connect between the lower medial side 21 and the medial fastening mechanism 37. As a result, the upper medial fastening end 17 and the lower medial fastening end 27 are rotatably attached to each other by the medial fastening mechanism 37. This configuration allows the user to change the medial angle between the thigh sleeve 1 and the calf sleeve 19 as the user's knee changes the angle.

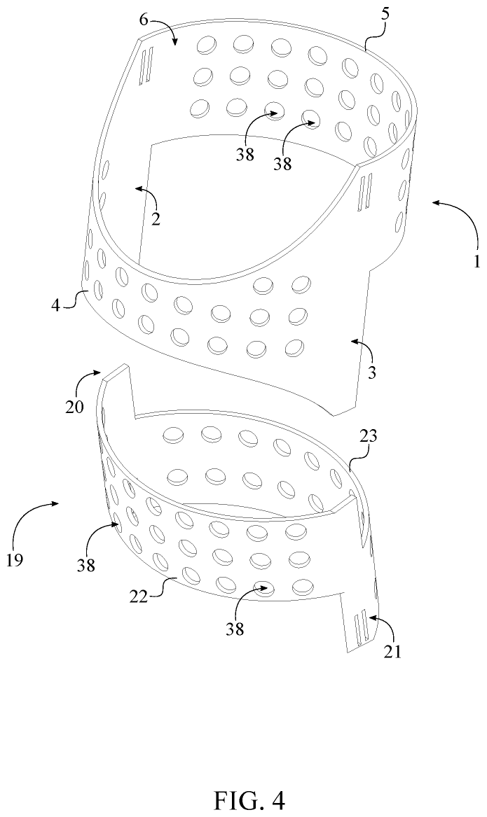

In reference to FIG. 4, the thigh sleeve 1 further comprises a front thigh cover 4, a rear thigh cover 5, and a thigh opening 6. The front thigh cover 4 and the rear thigh cover 5 are terminally connected to each other about the upper lateral side 2 and the upper medial side 3. This configuration ensures the front thigh cover 4 and the rear thigh cover 5 properly wrap around the front and the back of the user's thigh and forms the entire sleeve. The thigh opening 6 is delineated by the front thigh cover 4 and the rear thigh cover 5. The user may introduce the entire foot, knee, and part of the thigh through the thigh sleeve 1 via the thigh opening 6 until the appropriate part of the thigh is fitted between the front thigh cover 4 and the rear thigh cover 5.

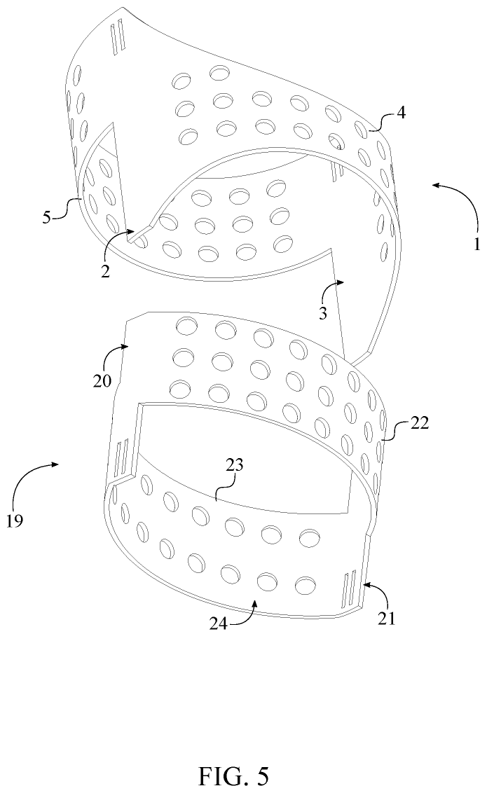

In reference to FIGS. 4 and 5, the calf sleeve 19 further comprises a front calf cover 22, a rear calf cover 23, and a calf opening 24. The front calf cover 22 and the rear calf cover 23 are terminally connected to each other about the lower lateral side 20 and the lower medial side 21. This configuration ensures the front calf cover 22 and the rear calf cover 23 properly wrap around the front and the back of the user's calf and forms the entire sleeve. The calf opening 24 is delineated by the front calf cover 22 and the rear calf cover 23. The user may introduce the entire foot and part of the calf through the calf sleeve 19 via the calf opening 24 until the appropriate part of the calf is fitted between the front calf cover 22 and the rear calf cover 23.

In reference to FIG. 3, the at least one thigh strap assembly 7 comprises a lateral strap 8, a medial strap 9, a female fastener 12, a male fastener 13, and an adjuster 14. The user may pull the lateral strap 8 to tighten the upper lateral side 2 to the lateral side of the user's thigh. The user may pull the medial strap 9 to tighten the upper medial side 3 to the medial side of the user's thigh. The lateral strap 8 and the medial strap 9 each comprise a proximal end 10 and a distal end 11. The proximal end 10 of the medial strap 9 is adjacently connected to the upper medial side 3, so as the user tightens up the medial strap 9, the proximal end 10 brings the upper medial side 3 closer to the user's thigh. The distal end 11 of the medial strap 9 is engaged through the adjuster 14. More specifically, the user may adjust the length of the medial strap 9 with the adjuster 14 as a mid-section of the medial strap 9 and the distal end 11 of the medial strap 9 are engaged within the adjuster 14. The proximal end 10 of the lateral strap 8 is connected to the upper lateral side 2, so as the user tightens up the lateral strap 8, the proximal end 10 brings the upper lateral side 2 closer to the user's thigh. The distal end 11 of the lateral strap 8 is connected to the male fastener 13. The medial strap 9 is looped through the female fastener 12. The user may join the lateral strap 8 and the medial strap 9 together by joining the male fastener 13 to the female fastener 12, then adjust the length of the strap via the adjuster 14 so both the upper medial side 3 and the upper lateral side 2 fit tightly to the user's thigh.

In reference to FIG. 3, the at least one calf strap assembly 25 comprises a lateral strap 8, a medial strap 9, a female fastener 12, a male fastener 13, and an adjuster 14. The user may pull the lateral strap 8 to tighten the lower lateral side 20 to the lateral side of the user's calf. The user may pull the medial strap 9 to tighten the lower medial side 21 to the medial side of the user's calf. The lateral strap 8 and the medial strap 9 each comprise a proximal end 10 and a distal end 11. The proximal end 10 of the medial strap 9 is adjacently connected to the lower medial side 21, so as the user tightens up the medial strap 9, the proximal end 10 brings the lower medial side 21 closer to the user's calf. The distal end 11 of the medial strap 9 is engaged through the adjuster 14. More specifically, the user may adjust the length of the medial strap 9 with the adjuster 14 as a mid-section of the medial strap 9 and the distal end 11 of the medial strap 9 are engaged within the adjuster 14. The proximal end 10 of the lateral strap 8 is connected to the lower lateral side 20, so as the user tightens up the lateral strap 8, the proximal end 10 brings the lower lateral side 20 closer to the user's calf. The distal end 11 of the lateral strap 8 is connected to the male fastener 13. The medial strap 9 is looped through the female fastener 12. The user may join the lateral strap 8 and the medial strap 9 together by joining the male fastener 13 to the female fastener 12, then adjust, the length of the strap via the adjuster 14 so both the lower medial side 21 and the lower lateral side 20 fit tightly to the user's calf.

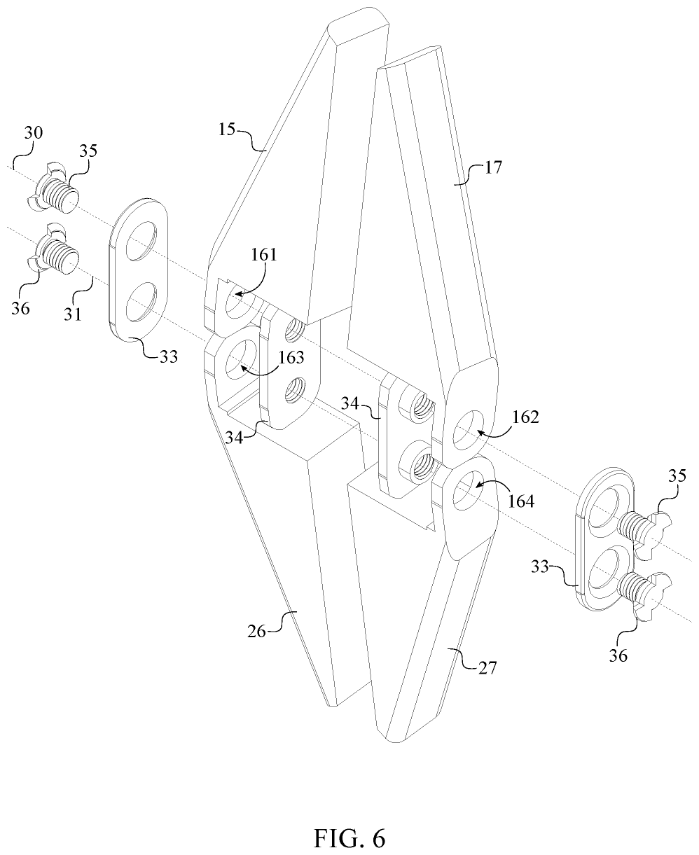

In reference to FIGS. 2 and 6, the present invention comprises a fastener opening 161, which traverses through the upper lateral fastening end 15, along an upper central axis 30. A fastener may be introduced into the fastener opening 161 to assemble the lateral fastening mechanism 32 with the upper lateral side 2. The lateral fastening mechanism 32 comprises an outer hinge plate 33, an inner hinge plate 34, and an upper fastener 35. The upper lateral fastening end 15 is positioned between the outer hinge plate 33 and the inner hinge plate 34 of the lateral fastening mechanism 32. By sandwiching the upper lateral fastening end 15 between the outer hinge plate 33 and the inner hinge plate 34 the present invention ensures the upper lateral fastening end 15 does not immediately detach from the lateral fastening mechanism 32 if either the outer hinge plate 33 or the inner hinge plate 34 is detached by accident. The upper fastener 35 of the lateral fastening mechanism 32 is removably engaged with the outer hinge plate 33, the fastener opening 161, and the inner hinge plate 34. In other words, the upper fastener 35 engages with the outer hinge plate 33, the fastener opening 161 and the inner hinge plate 34 and binds all three components together until the user removes the upper fastener 35 to disengages the upper lateral fastening end 15 from the lateral fastening mechanism 32.

In reference to FIGS. 2 and 6, the present invention comprises a fastener opening 162, which traverses through the upper medial fastening end 17, along an upper central axis 30. A fastener may be introduced into the fastener opening 162 to assemble the medial fastening mechanism 37 with the upper medial side 3. The medial fastening mechanism 37 comprises an outer hinge plate 33, an inner hinge plate 34, and an upper fastener 35. The upper medial fastening end 17 is positioned between the outer hinge plate 33 and the inner hinge plate 34 of the medial fastening mechanism 37 sandwiching the upper medial fastening end 47 between the outer hinge plate 33 and the inner hinge plate 34, the present invention ensures the upper medial fastening end 17 does not immediately detach from the medial fastening mechanism 37 if either outer hinge plate 33 or the inner hinge plate 34 is detached by accident. The upper fastener 35 of the medial fastening mechanism 37 is removably engaged with the outer hinge plate 33, the fastener opening 162, and the inner hinge plate 34. In other words, the upper fastener 35 engages with the outer hinge plate 33, the fastener opening 162, and the inner hinge plate 34 and binds all three components together until the user removes the upper fastener 35 to disengages the upper medial fastening end 17 from the medial fastening mechanism 37.

In reference to FIGS. 2 and 6, the present invention comprises a fastener opening 163, which traverses through the lower lateral fastening end 26, along a lower central axis 31. A fastener may be introduced into the fastener opening 163 to assemble the lateral fastening mechanism 32 with the lower lateral side 20. The lateral fastening mechanism 32 comprises an outer hinge plate 33, an inner hinge plate 34, and a lower fastener 36. The lower lateral fastening end 26 is positioned between the outer hinge plate 33 and the inner hinge plate 34 of the lateral fastening mechanism 32. By sandwiching the lower lateral fastening end 26 between the outer hinge plate 33 and the inner hinge plate 34, the present invention ensures the lower lateral fastening end 26 does not immediately detach from the lateral fastening mechanism 32 if either outer hinge plate 33 or the inner hinge plate 34 is detached by accident. The lower fastener 36 of the lateral fastening mechanism 32 is removably engaged with the outer hinge plate 33, the fastener opening 163, and the inner hinge plate 34. In other words, the lower fastener 36 engages with the outer hinge plate 33, the fastener opening 163, and the inner hinge plate 34 and binds all three components together until the user remove the lower fastener 36 to disengages the lower lateral fastening end 26 from the lateral fastening mechanism 32.

In reference to FIGS. 2 and 6, the present invention comprises a fastener opening 164, which traverses through the lower medial fastening end 27, along a lower central axis 31. A fastener may be introduced into the fastener opening 164 to assemble the medial fastening mechanism 37 with the lower medial side 21. The medial fastening mechanism 37 comprises an outer hinge plate 33, an inner hinge plate 34, and a lower fastener 36. The lower medial fastening end 27 is positioned between the outer hinge plate 33 and the inner hinge plate 34 of the medial fastening mechanism 37. By sandwiching the lower medial fastening end 27 between the outer hinge plate 33 and the inner hinge plate 34, the present invention ensures the lower medial fastening end 27 does not immediately detach from the medial fastening mechanism 37 if either outer hinge plate 33 or the inner hinge plate 34 is detached by accident. The lower fastener 36 of the medial fastening mechanism 37 is removably engaged with the outer hinge plate 33, the fastener opening 164, and the inner hinge plate 34. In other words, the lower fastener 36 engages with the outer hinge plate 33, the fastener opening 164, and the inner hinge plate 34 and binds all three components together until the user removes the upper fastener 35 to disengages the lower medial fastening end 27 from the medial fastening mechanism 37.

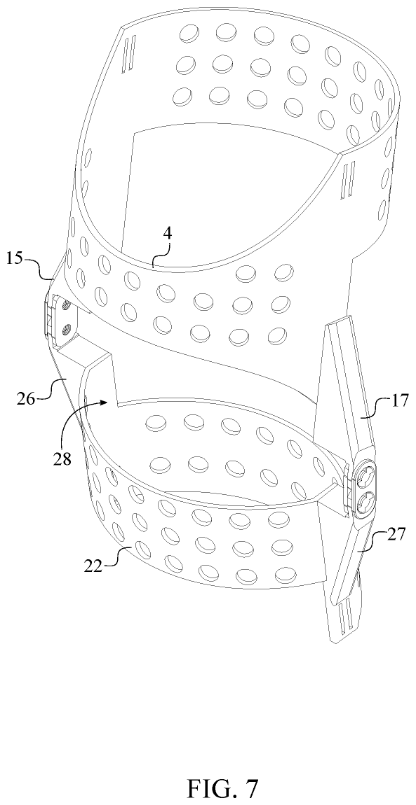

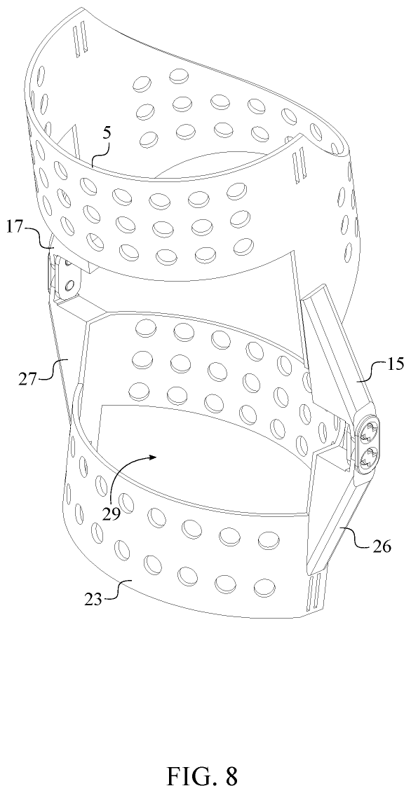

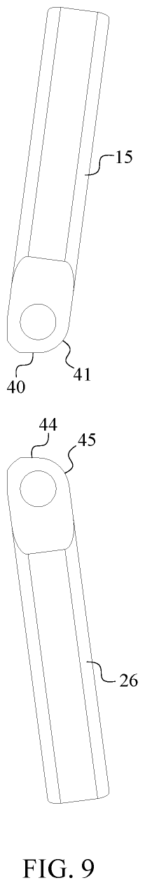

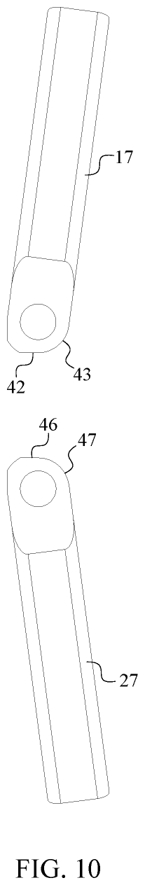

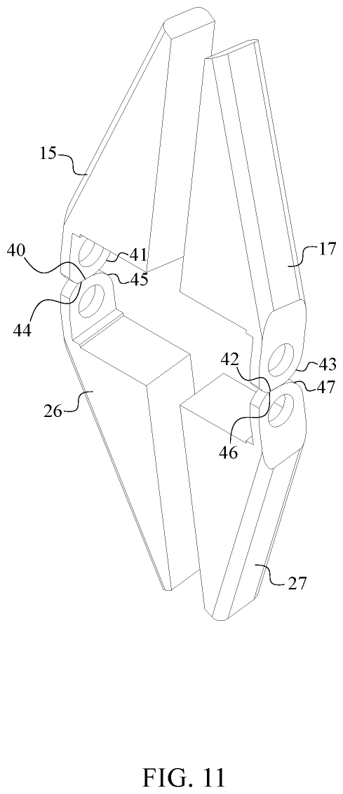

In reference to FIGS. 7 and 8, the present invention comprises a knee opening 28, which is delineated by the front thigh cover 4, the upper medial fastening end 17, the lower medial fastening end 27, the front calf cover 22, the lower lateral fastening end 26, and the upper lateral fastening end 15. After the user pull the thigh sleeve 1 to the appropriate position on the thigh, the user may introduce the knee to the knee opening 28, so the present invention does not cover the front of the knee. The present invention comprises a joint opening 29, which is delineated by the rear thigh cover 5, the upper medial fastening end 17, the lower medial fastening end 27, the rear calf cover 23, the lower lateral fastening end 26, and the upper lateral fastening end 15. After the user pull the calf sleeve 19 to the appropriate position on the calf, the user may introduce the joint to the joint opening 29, so the present invention does not cover the back of the knee in reference to FIG. 9-11, the upper lateral fastening end 15 comprises an upper lateral flat surface 40 and an upper lateral curved surface 41. The upper lateral flat surface 40 is adjacently positioned to the upper lateral curved surface 41, but the upper lateral curved surface 41 is positioned closer to the back of the knee. The upper medial fastening end 17 comprises an upper medial flat surface 42 and an upper medial curved surface 43. The upper medial flat surface 42 is adjacently positioned to the upper medial curved surface 43, but the upper medial curved surface 43 is positioned closer to the back of the knee. The lower lateral fastening end 26 comprises a lower lateral flat surface 44 and a lower lateral curved surface 45. The lower lateral flat surface 44 is adjacently positioned to the lower lateral curved surface 45, but the lower lateral curved surface 45 is positioned closer to the hack of the knee. The lower medial fastening end 27 comprises a lower medial flat surface 46 and a lower medial curved surface 47. The lower medial flat surface 46 is adjacently positioned to the lower medial curved surface 47, but the lower medial curved surface 47 is positioned closer to the back of the knee.

In reference to FIG. 9-11, the upper lateral flat surface 40 and the lower lateral flat surface 44 are positioned parallel to each other when the user's leg is totally straight. This configuration is to ensure the thigh sleeve 1 and the calf sleeve 19 do not hyperextend the user's knee about the lateral side of the knee thus maintaining the user's leg in a straight position by locking the upper lateral flat surface 40 to the lower lateral flat surface 44. The upper medial flat surface 42 and the lower medial flat surface 46 are positioned parallel to each other when the user's leg is straight. This configuration is to ensure the thigh sleeve 1 and the calf sleeve 19 do not hyperextend the user's knee about the medial side of the knee thus maintaining the user's leg in a straight position by locking the upper medial flat surface 42 to the lower medial flat surface 46.

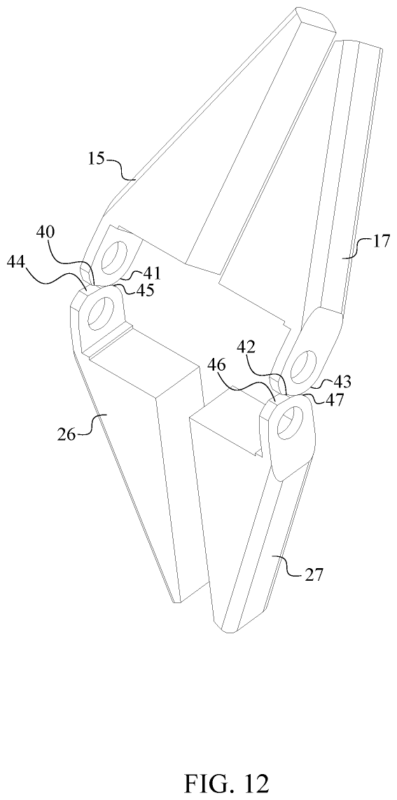

In reference to FIGS. 9, 10, and 12, the upper lateral curved surface 41 is tangentially pressed against the lower lateral curved surface 45, which unlocks the upper lateral fastening end 15 from the lower lateral fastening end 26, as the user's calf and thigh changes from a straight position to a bent position, such as sitting down. This configuration is to ensure the lateral fastening mechanism 32 does not hinder user's movements about the lateral side of the knee. The upper medial curved surface 43 is tangentially pressed against the lower medial curved surface 47, which unlocks the upper medial fastening end 17 from the lower medial fastening end 27, as the user's calf and thigh changes from a straight position to a bent position, such as sitting down. This configuration is to ensure the lateral fastening mechanism 32 does not hinder user's movements about the medial side of the knee.

In reference to FIG. 4, both the calf sleeve 19 and the thigh sleeve 1 comprise a plurality of ventilation openings 38, which traverses through both calf sleeve 19 and the thigh sleeve 1. The plurality of ventilation openings 3 is configured to facilitate airflow between the user's skin and the calf sleeve 19 and thigh sleeve 1.

Although the invention has been explained in relation to its preferred embodiment, it is to be understood that many other possible modifications and variations can be made without departing from the spirit and scope of the invention.

What is claimed is:

1. A knee brace comprises:
a thigh sleeve;
a calf sleeve;
a lateral fastening mechanism;
a medial fastening mechanism;
the thigh sleeve comprising an upper lateral side, an upper medial side, at least one thigh strap assembly, an upper lateral fastening end, and an upper medial fastening end;
the calf sleeve comprising a lower lateral side, a lower medial side, at least one calf strap assembly, a lower lateral fastening end, and a lower medial fastening end;
the at least one thigh strap assembly being adjustably integrated onto the upper lateral side and the upper medial side;
the at least one calf strap assembly being adjustably integrated onto the lower lateral side and the lower medial side;
the upper lateral fastening end being terminally connected onto the upper lateral side;
the upper medial fastening end being terminally connected onto the upper medial side;
the lower lateral fastening end being terminally connected onto the lower lateral side;
the lower medial fastening end being terminally connected onto the lower medial side;
the upper lateral fastening end and the lower lateral fastening end being rotatably attached to each other by the later fastening mechanism; and
the upper medial fastening end and the lower medial fastening end being rotatably attached to each other by the medial fastening mechanism.

2. The knee brace as claimed in claim 1 comprises:
the thigh sleeve further comprising a front thigh cover, a rear thigh cover, and a thigh opening;
the front thigh cover and the rear thigh cover being terminal connected to each other about the upper lateral side and the upper medial side; and
the thigh opening being delineated by the front thigh cover and the rear thigh cover.

3. The knee brace as claimed in claim 1 comprises:
the calf sleeve further comprising a front calf cover, a rear calf cover, and a calf opening;
the front calf cover and the rear calf cover being terminally connected to each other about the lower lateral side and the lower medial side; and
the calf opening being delineated by the front calf cover and the rear calf cover.

4. The knee brace as claimed in claim 1 comprises:
the at least one thigh strap assembly comprising a lateral strap, a medial strap, a female fastener, a male fastener, and an adjuster;
the lateral strap and the medial strap each comprising a proximal end and a distal end;
the medial strap being looped through the female fastener;
the distal end of the medial strap being engaged through the adjuster;
the proximal end of the medial strap being adjacently connected to the upper medial side;
the distal end of the lateral strap being connected to the male fastener; and
the proximal end of the lateral strap being connected to the upper lateral side.

5. The knee brace as claimed in claim 1 comprises:
the at least one calf strap assembly comprising a lateral strap, a medial strap, a female fastener, a male fastener, and an adjuster;
the lateral strap and the medial strap each comprising a proximal end and a distal end;
the medial strap being looped through the female fastener;
the distal end of the medial strap being engaged through the adjuster;
the proximal end of the medial strap being adjacently connected to the lower medial side;
the distal end of the lateral strap being connected to the male fastener; and
the proximal end of the lateral strap being connected to the lower lateral side.

6. The knee brace as claimed in claim 1 comprise:
a fastener opening;
the fastener opening traversing through the upper lateral fastening end,
along an upper central axis;
the lateral fastening mechanism comprising an outer hinge plate, an inner hinge plate, and an upper fastener;
the upper lateral fastening end being positioned between the outer hinge plate and the inner hinge plate of the lateral fastening mechanism; and
the upper fastener of the lateral fastening mechanism being removably engaged with the outer hinge plate, the fastener opening, and the inner hinge plate.

7. The knee brace as claimed in claim 1 comprises
a fastener opening;
the fastener opening traversing through the upper medial fastening end, along an upper central axis;
the medial fastening mechanism comprising an outer hinge plate, an inner hinge plate, and an upper fastener;
the upper medial fastening end being positioned between the outer hinge plate and the inner hinge plate of the medial fastening mechanism; and
the upper fastener of the medial fastening mechanism being removably engaged with the outer hinge plate, the fastener opening, and the inner hinge plate.

8. The knee brace as claimed in claim 1 comprise:
a fastener opening;
the fastener opening traversing through the lower lateral fastening end, along a lower central axis;
the lateral fastening mechanism comprising an outer hinge plate, an inner hinge plate, and a lower fastener;
the lower lateral fastening end being positioned between the outer hinge plate and the inner hinge plate of the later fastening mechanism; and
the lower fastener of the lateral fastening mechanism being removably engaged with through the outer hinge plate, the fastener opening, and the inner hinge plate.

9. The knee brace as claimed in claim 1 comprise:
a fastener opening;
the fastener opening traversing through the lower medial fastening end, along a lower central axis;
the medial fastening mechanism comprising an outer hinge plate, an inner hinge plate, and a lower fastener;
the lower lateral fastening end being positioned between the outer hinge plate and the inner hinge plate of the medial fastening mechanism; and
the lower fastener of the medial fastening mechanism being removably engaged with through the outer hinge plate, the fastener opening, and the inner hinge plate.

10. The knee brace as claimed in claim 1, comprise:
a knee opening;
the thigh sleeve comprising a front thigh cover;
the calf sleeve comprising a front calf cover; and
the knee opening being delineated by the front thigh cover, the upper medial fastening end, the lower medial fastening end, the front calf cover, the lower lateral fastening end, and the upper lateral fastening end.

11. The knee brace as claimed in claim 1 comprise:
a joint opening;
the thigh sleeve comprising a rear thigh cover;
the calf sleeve comprising a rear calf cover; and
the joint opening being delineated by the rear thigh cover, the upper medial fastening end, the lower medial fastening end, the rear calf cover, the lower lateral fastening end, and the upper lateral fastening end.

12. The knee brace as claimed in claim 1 comprise:
the upper lateral fastening end comprising an upper lateral flat surface and an upper lateral curved surface;
the upper medial fastening end comprising an upper medial flat surface and an upper medial curved surface;
the lower lateral fastening end comprising a lower lateral flat surface and a lower lateral curved surface;
the lower medial fastening end comprising a lower medial flat surface and a lower medial curved surface;
the upper lateral flat surface being adjacently positioned to the upper lateral curved surface;
the upper medial flat surface being adjacently positioned to the upper medial curved surface;
the lower lateral flat surface being adjacently positioned to the lower lateral curved surface; and
the lower medial flat surface being adjacently positioned to the lower medial curved surface.

13. The knee brace as claimed in claim 12 comprise:
the upper lateral fiat surface and the lower lateral flat surface being positioned parallel to each other; and
the upper medial flat surface and the lower medial flat surface being positioned parallel to each other.

14. The knee brace as claimed in claim 12 comprise:
the upper lateral curved surface being tangentially pressed against the lower lateral curved surface; and
the upper medial curved surface being tangentially pressed against the lower medial curved surface

15. The knee brace as claimed in claim 1 comprise:
a plurality of ventilation openings; and
the plurality of ventilation openings traversing through the thigh sleeve and the calf sleeve.
